(19) United States
(12) Patent Application Publication
(10) Pub. No.: US 2012/0071913 A1
(43) Pub. Date: Mar. 22, 2012

(54) NASAL SPLINTS

(76) Inventor: Oscar A. TAMEZ, Georgetown, TX (US)

(21) Appl. No.: 12/887,108

(22) Filed: Sep. 21, 2010

(51) Int. Cl. A61M 29/00

(52) U.S. Cl. 606/199

(57) ABSTRACT

One embodiment of the present application relates to a system including a nasal splint and an insert. The nasal splint has an airway tube and the insert includes an elongated tube for insertion into the airway tube of the nasal splint. The elongated tube of the insert is configured to collect an obstruction of human material while installed in the airway tube and to remove the obstruction when the insert is removed from the airway tube.

### BACKGROUND

The present invention relates generally to the field of nasal splints.

Nasal splints are often used to stabilize the septum of the nose during a healing process after ear, nose, or throat surgery involving the septum. Conventional nasal splints, such as those shown and described in U.S. Pat. No. 3,935,859 to Doyle, include airway tubes. The airway tubes can become clogged with mucous or blood, resulting in nasal obstruction and trouble breathing for the patient. It is challenging and difficult to develop nasal splints that are acceptable for the surgeon's use and comfortable or easy to use for the patient.

### SUMMARY

One embodiment of the present invention relates to a system including a nasal splint and an insert. The nasal splint has an airway tube and the insert includes an elongated tube for insertion into the airway tube of the nasal splint. The elongated tube of the insert is configured to collect an obstruction of human material while installed in the airway tube and to remove the obstruction when the insert is removed from the airway tube.

Another embodiment of the present invention relates to a system including a nasal splint and an insert for insertion into a patient's nose with the nasal splint. The insert has an elongated tube for providing the patient with an airway passage while the nasal splint and the insert are installed in the patient's nose. The elongated tube is configured to remove an obstruction of human material from the patient's nose when the insert is removed from the patient's nose and the nasal splint remains in the patient's nose.

Yet another embodiment of the present invention relates to an insert for use with a nasal splint. The insert includes an elongated tube for insertion into a patient's nose with the nasal splint. The insert further includes a plurality of protrusions extending from the interior of the elongated tube and into an airway passage formed by the elongated tube.

Another embodiment of the present invention relates to a system including a nasal splint and an insert for the nasal splint. The nasal splint includes an airway tube. The insert includes an elongated tube for insertion into the airway tube of the nasal splint. The elongated tube has an exterior surface that presses against the airway tube and an interior surface that forms an airway passage. The insert further includes a plurality of protrusions extending from the interior surface of the elongated tube and into the airway passage.

Another embodiment of the present invention relates to a device for use with a nasal splint having an airway tube. The device includes an elongated tube for insertion into the airway tube of the nasal splint, the elongated tube having an exterior surface that presses against the airway tube and an interior surface that forms an airway passage. The device further includes a plurality of protrusions extending from the interior surface of the elongated tube and into the airway passage.

Yet another embodiment of the present invention relates to a method for treating a patient. The method includes inserting a nasal splint into a patient's nose, the nasal splint including an airway tube. The method further includes inserting an insert into the airway tube, the insert including an elongated tube and providing an airway passage for the patient within the airway tube. The method also includes allowing an obstruction of human material to form within the airway passage. The method yet further includes removing the insert from the nasal splint and the patient's nose to remove the obstruction of human material from the patient's nose with the insert. The elongated tube may include a plurality of protrusions for carrying the obstruction with the insert when the insert is removed from the airway tube of the nasal splint. The method may further include inserting another insert into the nasal splint after the insert with the obstruction of human material is removed from the nasal splint. It may be possible to repeat the insertion and removing steps while human material continues to be collected by the inserts. A variation of the method comprises inserting a nasal splint into a patient's nose, which nasal splint is equipped with the inventive insert. The insert may be removed later to remove an obstruction from an airway passage. A second insert may then optionally be installed. Thus the invention contemplates a kit that includes a pair of nasal splits each member of the pair equipped with an insert, plus at least one set of replacement inserts.

Another embodiment of the present invention relates to system that includes a nasal pack having an airway tube. The system also includes an insert. The insert includes an elongated tube for insertion into the airway tube of the nasal pack. The elongated tube has an exterior surface that presses against the airway tube and an interior surface that forms an airway passage. The insert further includes a plurality of protrusions extending from the interior surface of the elongated tube and into the airway passage.

Yet another embodiment of the present invention relates to a method for treating a patient having a nasal splint installed in his or her nose, the nasal splint including an airway tube. The method includes inserting an insert into the airway tube, the insert comprising an elongated tube and providing an airway passage for the patient within the airway tube. The insert is configured to remove an obstruction of human material from the airway tube when the insert is removed from the airway tube.

Alternative exemplary embodiments relate to other features and combinations of features as may be generally recited in the claims.

### BRIEF DESCRIPTION OF THE FIGURES

The disclosure will become more fully understood from the following detailed description, taken in conjunction with the accompanying figures, wherein like reference numerals refer to like elements, in which:

FIG. 1 is a view of a nasal splint system with an insert beside a nasal splint, according to an exemplary embodiment;

FIG. 2 is a view of the nasal splint system of FIG. 1 with the insert shown as installed within the nasal splint, according to an exemplary embodiment;

FIG. 3 is a side view of the nasal splint of FIG. 1, according to an exemplary embodiment;

FIG. 4 is a side view of the nasal splint of FIG. 2 with the insert shown as installed, according to an exemplary embodiment;

FIG. 5 is a perspective view of the nasal splint insert shown in FIGS. 1, 2, and 4, according to an exemplary embodiment;

FIG. 6 is a top down view of the back side of an improved nasal splint for use with nasal splint inserts as described herein, according to an exemplary embodiment;

FIG. 7 is a view of an improved nasal splint system for use with nasal splint inserts as described herein, according to another exemplary embodiment; and

FIG. 8 is a view of a nasal pack system including an insert according to yet another exemplary embodiment.

### DETAILED DESCRIPTION OF EXEMPLARY EMBODIMENTS

Before turning to the figures, which illustrate the exemplary embodiments in detail, it should be understood that the disclosure is not limited to the details or methodology set forth in the description or illustrated in the figures. It should also be understood that the terminology is for the purpose of description only and should not be regarded as limiting.

Referring generally to the Figures, an insert having an elongated tube is inserted into the airway tube of a nasal splint. After an obstruction of blood, mucus, or other fluids forms within the elongated tube of the insert, the insert is removed from the airway tube, taking the obstruction with it and clearing the nasal splint's airway tube. The system of the nasal splint and the insert described herein may advantageously allow for a patient to clear an airway obstruction without assistance.

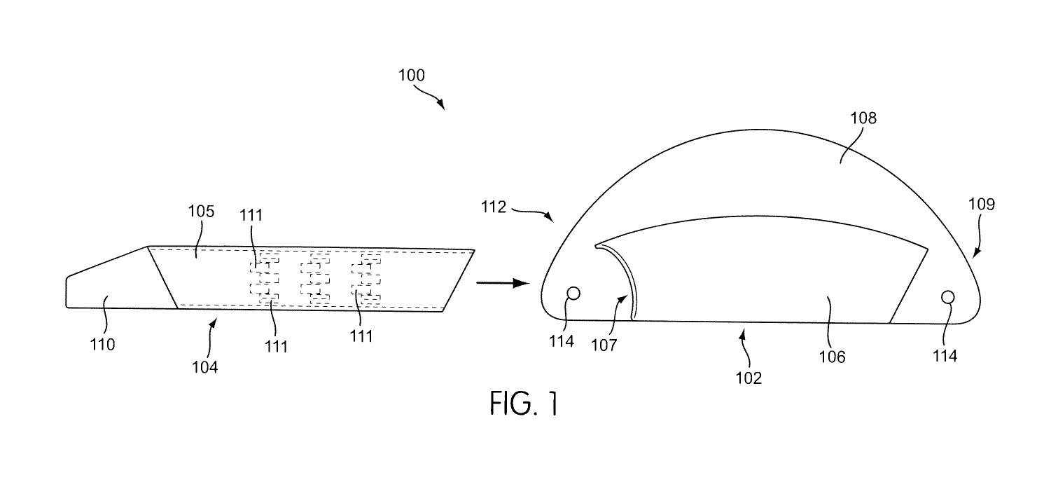

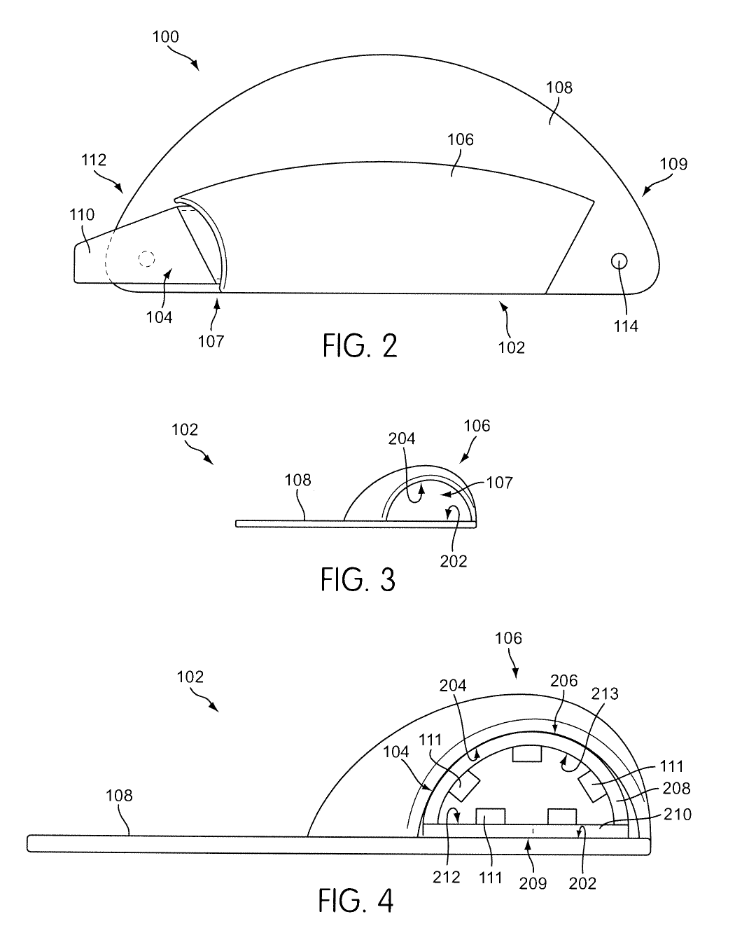

Referring now to FIG. 1, a perspective view of a nasal splint system 100 is shown, according to an exemplary embodiment. Nasal splint system 100 includes a nasal splint 102 and an insert 104. Nasal splint 102 includes an airway tube 106. Nasal splint 102 is an intranasal airway splint including a septum plate 108. Septum plate 108 supports airway tube 106. Insert 104 includes an elongated tube 105. Insert 104 including elongated tube 105 is inserted into an opening 107 of airway tube 106. FIG. 2 illustrates insert 104 as fully inserted within airway tube 106 of nasal splint 102. When insert 104 is housed within airway tube 106 and when nasal splint 102 is held inside the nasal cavity, the patient may breathe through elongated tube 105 of insert 104. Elongated tube 105 may fill with mucus or blood as the patient heals, impacting the patient's ability to breathe through his or her nose. An obstruction of mucus or blood can be removed from nasal splint 102 by removing insert 104 from nasal splint 102. Insert 104 can include one or more protrusions 111 extending from the interior surface of elongated tube 105 and into the airway passage formed by elongated tube 105. Protrusions 111 can be used to help extract the obstruction of mucus and blood by helping to snag or “grab” the obstruction of mucus or blood as insert 104 is being removed from airway tube 106. Protrusions 111 are intended to prevent the obstruction of blood and mucus from separating from insert 104 when insert 104 is removed from airway tube 106.

Insert 104 can advantageously be removed by a patient to clear an obstruction of blood or mucus. As an obstruction of blood and mucus is most likely to form at the beginning of a recovery period, self-removal of the obstruction via removal of insert 104 can advantageously allow a patient to enjoy an easier recovery period prior to his or her next appointment with a healthcare professional (e.g., prior to the time when nasal splint 102 itself can be removed).

In an exemplary embodiment, insert 104 is sized to fit inside the airway tube of different nasal splints via only a friction fit between the exterior surface of insert 104 and the inside surface of nasal splint 102's airway tube 106. In another exemplary embodiment, insert 104 does not fit inside the airway tube of a nasal splint via only a friction fit and is sutured, clamped, adhered, taped or otherwise mechanically held inside the nasal splint airway tube.

The nasal splint for use with insert 104 can be any type of nasal splint having an airway tube through which the patient is intended to breathe while the nasal splint is inserted in the patient's nose. In the embodiment shown in FIG. 1, nasal splint 102 is a “Tellez” type nasal splint. In other embodiments the nasal splint can be a “Doyle” type nasal splint, a “Doyle II” type nasal splint, an “Eliachar” type nasal splint, or a “Goldsmith Inflatable” type nasal splint. The nasal splint can be installed after septum repair, nasal fracture repair, septal perforation repair, skull based surgery via intranasal approach, extensive degloving repair of the nose for cancer or other trauma of the nose, cosmetic or functional rhinoplasty, or other operations. In yet other embodiments the nasal splint can be of a different type or design of the past, present, or future including an airway tube. For example, some nasal splints stint the sinus cavities and include an airway tube for the purpose of allowing air flow within the sinus cavities. Inserts as described herein can be used with the airway tubes of such nasal splints, modified for size and/or shape to appropriately fit sinus cavity nasal splints.

Nasal splint 102 can be a one-piece device formed from a flexible and biocompatible material such as medical grade silicon. Nasal splint 102 can also, or alternatively, be formed from other materials. For example, nasal splint 102 can be formed from a flexible plastic material such as polyvinyl chloride, fluoroplastic, rubber, or another material or combination of materials. The material or combination of materials for nasal splint 102 can be chosen to provide sufficient rigidity to perform the intended splint function. It should be appreciated that different nasal splint designs or different nasal splint materials can be utilized depending on the size of the patient, intended application, allergies of the patient, or other considerations. The nasal splint can be as described in U.S. Pat. No. 3,935,859, issued Feb. 3, 1976 or as described in U.S. Pat. No. 5,139,510, issued Aug. 18, 1992. In other embodiments, different designs, sizes, or shapes of nasal splints can be utilized with an insert of the present disclosure. For example, a multi-piece nasal splint having an airway tube 106 that attaches or fits next to a septum plate or septum balloon can be utilized with an insert of the present disclosure. For example, in one alternative embodiment, an insert of the present disclosure can be used with a tubeless nasal splint such as that shown in U.S. Pat. No. D468,826, issued Jan. 14, 2003. In such an embodiment, for example, the insert can be sized and shaped to rest against structures of the nasal splint while the nasal splint is installed within a patient's nose. When an obstruction forms, the patient or a healthcare professional removes the insert while leaving the tubeless nasal splint in the patient's nose.

Referring still to FIG. 1, holes 114 are shown in nasal splint 102. One or both of holes 114 can be used to suture nasal splint 102 in place inside the nose. In some instances only one of holes 114 will be used to secure nasal splint 102 to the nose. For example, if splint end 109 is inserted into the patient's nose first, hole 114 at splint end 112 might be the only hole used to suture nasal splint 102 to the nose.

Referring now to FIG. 2, insert 104 is shown as installed in airway tube 106. Insert 104 is shown to include a tab 110 extending from one end of elongated tube 105. In an exemplary embodiment, tab 110 can be grabbed and pulled by a patient to remove insert 104 from airway tube 106 particularly and nasal splint 102 generally. While tab 110 is shown as being integrally formed from the material of insert 104 and shaped as a wide portion for a user to grip between his or her fingers, in other exemplary embodiments tab 110 may be shaped or sized differently. For example, in some embodiments a relatively thin string, strip, or other extension may connect to insert 104 or elongated tube 105 to provide a mechanism for allowing a patient to pull insert 104 relative to nasal splint 102. In yet other embodiments insert 104 does not include tab 110. In such embodiments, insert 104 and elongated tube 105 may be sized to be longer than airway tube 106 such that the end of insert 104 and elongated tube 105 extend beyond opening 107 and out of the patient's nose. In those embodiments, when the time comes to remove insert 104, the extended end of insert 104 or elongated tube 105 can be gripped and pulled on by the patient or a healthcare professional to remove insert 104.

Referring now to FIG. 3, a side profile view of nasal splint 102 without an insert installed is shown, according to an exemplary embodiment. Septum plate 108 is shown as being a flat piece of material from which airway tube 106 extends on one side. In other embodiments, airway tube 106 may be formed by septum plate 108 rolling in or over on itself to form a tube or a tube-like structure. In the embodiments shown in the Figures, airway tube 106 is semi-cylindrical, including a curved portion and a flat portion, with interior surface 204 corresponding to the curved portion and interior surface 202 corresponding to the flat portion of airway tube 106.

Referring now to FIG. 4, a side profile view of nasal splint 102 having insert 104 installed in airway tube 106 is shown, according to an exemplary embodiment. Insert 104 is sized and shaped to match the interior shape of airway tube 106. Exterior surfaces 206, 209 of insert 104 press against interior surfaces 202, 204 of airway tube 106. In the embodiments shown in the Figures, insert 104 is semi-cylindrical and approximately matches the size and shape of airway tube 106. For example, insert 104's exterior surfaces 206, 209 correspond to a curved portion 208 and a flat portion 210 (respectively) of airway tube 106. In alternative embodiments, insert 104 may be sized and shaped differently than airway tube 106. For example, insert 104 can be round. Insert 104 may have a diameter that is the same as or larger than at least one of the dimensions of opening 107, creating a friction fit between an exterior surface of insert 104 and at least one interior surface of airway tube 106. In yet other embodiments, interior surfaces 202, 204 of airway tube 106 may include one or more protrusions, rough areas, texture areas, or other structures for improving the friction fit with exterior surfaces 206, 209 of insert 104. In still other embodiments, interior surfaces 202, 204 of airway tube may be smoothed or coated with a friction reducing material to facilitate easy installation or removal of insert 104.

Referring still to FIG. 4, protrusions 111 are shown to extend from the interior surfaces 212, 213 of insert 104 and into the airway passage created when insert 104 is installed within airway tube 106. As shown in FIG. 4 (and FIG. 1), protrusions 111 can be small rectangular pieces of material extending from both interior surface 212 corresponding with the flat portion of insert 104 and interior surface 213 corresponding with the curved portion of insert 104. In other embodiments, protrusions 111 can extend from one but not both of interior surface 212 and interior surface 213. Protrusions 111 can be integrally formed with insert 104 or surfaces 212, 213 or protrusions 111 can be separate pieces adhered, bonded, or otherwise coupled to insert 104 or elongated tube 105. While protrusions 111 are shown as being substantially uniform in dimension, in other embodiments protrusions 111 can be of varying shapes, sizes, or depths. Further, while protrusions 111 are shown as being orderly and consistently spaced apart from one another, in other embodiments protrusions 111 can be randomly spaced, semi-randomly spaced, spaced apart in an odd pattern, or otherwise.

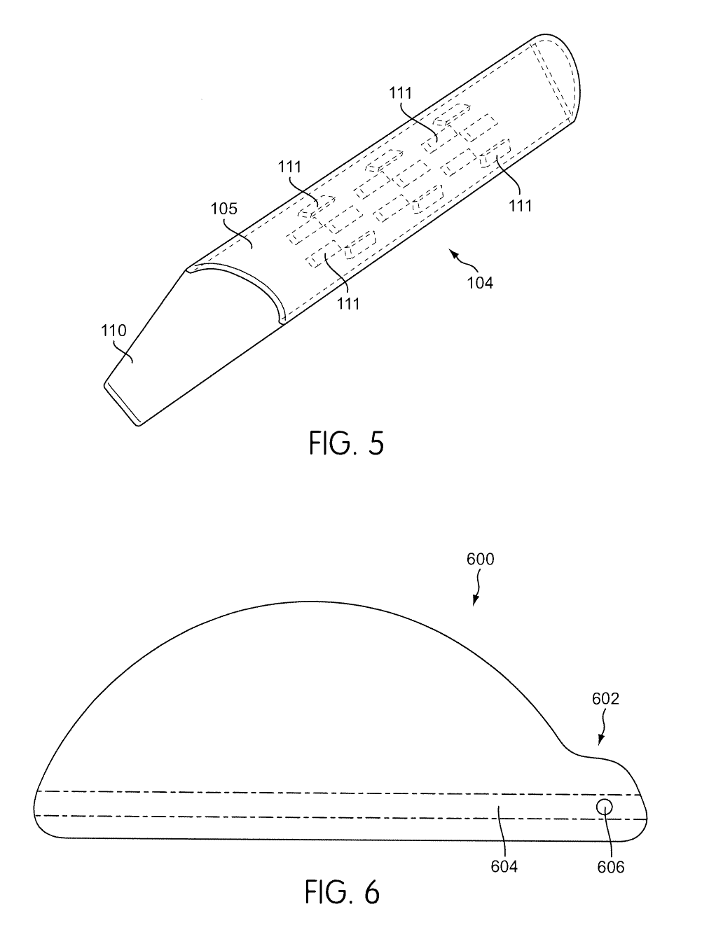

FIG. 5 illustrates a perspective view of insert 104, according to an exemplary embodiment. The interior of elongated tube 105 is shown in broken lines to illustrate that the material of elongated tube 105 can be transparent, translucent, or semi-transparent. In other embodiments, elongated tube 105 may not be transparent, translucent, or semi-transparent. FIG. 5 also provides another view of the shape and arrangement of protrusions 111, according to an exemplary embodiment. In the Figures, the plurality of protrusions 111 are shown as arranged in a plurality of columns down the length of elongated tube 105. In some alternative embodiments, the plurality of protrusions may be arranged in a single column down the length of the elongated tube of the insert. In the Figures, the plurality of protrusions 111 are also shown as arranged in a plurality of rows across the length of elongated tube 105. In some alternative embodiments, the plurality of protrusions may be arranged in a single row across the length of the elongated tube of the insert. As shown in the Figures, the plurality of protrusions 111 are arranged in at least one column down the length of elongated tube 105 and are arranged in at least one row across the length of elongated tube 105. While the columns and rows of protrusions are shown as being relatively parallel or uniformly aligned in the Figures, in other exemplary embodiments the columns or rows may be intersecting or staggered relative to each other. As shown in FIG. 5, the columns and rows of protrusions 111 along the flat portion of elongated tube 105 are located at a first set of offsets relative to the length and width of elongated tube 105 while the columns and rows of protrusions 111 along the curved portion of elongated tube 105 are located at a second set of offsets relative to the length and width of elongated tube 105. As shown in FIG. 5, the offsets of the protrusions 111 may form a staggered pattern between the protrusions on the flat portion of elongated tube 105 and the curved portion of elongated tube 105. The offsets and resulting staggered pattern shown in FIG. 5 can advantageously provide for an improved hold on blood or mucus obstructions by protrusions 111.

In FIG. 5, protrusions 111 are shown as being of relatively flat shapes with relatively flat sides and edges and of relatively uniform dimensions. According to an exemplary embodiment, each protrusion 111 is at least one millimeter high relative to the interior surface of the elongated tube. Further, each protrusion 111 is at least one millimeter high, one millimeter wide, and one millimeter long. Yet further, the plurality of protrusions 111 are spaced apart by at least one millimeter.

In alternative embodiments, protrusions 111 can be of varying heights, widths, or lengths. Further, adjacent protrusions can be shaped differently. Yet further, some protrusions of elongated tube 105 can be loops or hooks such as provided by VELCRO style hook and loop pieces. In other embodiments, the protrusions of elongated tube 105 take the form of spikes, bristles, or rough raised portions of the interior surface of the elongated tube. 19. In varying embodiments, the protrusions can be or include a raised matrix or a raised grid having cells, pores or honeycomb structures that can catch and carry human material (e.g., blood, mucus, etc.). In yet other embodiments, a combination of two or more types of protrusions can exist within the insert. For example, the cells, pores or honeycomb structures might be provided to catch relatively fluid human material while hooks, spikes or bristles might be provided to catch material that has dried or that may not flow into the cells, pores or honeycomb structures.

Insert 104 can be made from the same material as nasal splint 102. In other embodiments, insert 104 is made from a different material. The material of insert 104 can be selected to create a friction fit with the interior surfaces of the nasal splint 102's airway tube 106. For example, if an ultra-smooth material or surface treatment is applied to the surfaces of nasal splint 102 and airway tube 106, the material of insert 104 may be of a different material expected to hold within airway tube 106 via friction when an exterior surface of insert 104 presses against an interior surface of airway tube 106.

Referring now to FIG. 6, an improved nasal splint 600 for use with a nasal splint insert such as insert 104 is shown, according an exemplary embodiment. FIG. 6 is an illustration of the back side of nasal splint 600 (i.e., the flat side of the septum plate). An airway tube similar to the airway tube 106 shown in FIGS. 1-4 can be located on the opposite side of nasal splint 600. Nasal splint 600 can advantageously facilitate easy removal of an insert from nasal splint 600's airway tube. Accordingly, nasal splint 600 is intended to allow a patient to remove the insert without the assistance of a healthcare professional. To make nasal splint 600 more accessible to the patient's fingers (or tweezers, pliers, or another mechanical tool), an extended portion 602 projects from one end of nasal splint 600. In the embodiment shown in FIG. 6, the extended portion 602 extends from the anterior end of the nasal splint 600 (i.e., the end that is closer to the front of the patient's nose). Extended portion 602 is shown to include a hole 606 for a suture. Hole 606 may be used to suture tab 110 of insert 104 (shown in previous Figures) to extended portion 602 of nasal splint 600 and/or may be used to suture extended portion 602 to the patient's nose. In use, the patient can pinch or grab extended portion 602 with one hand while pulling on tab 110 of insert 104 with the other hand. Extended portion 602 can advantageously prevent the patient from pulling on nasal splint 600 when removing insert 104.

Depending on the friction or fit between the insert and the airway tube of the nasal splint, pulling on the insert might cause the splint or the airway tube to buckle when the insert is being removed. Nasal splint 600 advantageously includes a support 604 to stabilize the splint so that the splint or the airway tube does not buckle (or does not buckle too extremely) when the insert is being removed from splint 600. In an exemplary embodiment support 604 is an aluminum strip embedded within the material of nasal splint 600. Support 604 is shown to extend into extended portion 602. In the embodiment shown in FIG. 6, extended portion 602 and support 604 are intended to operate in concert to provide a stable and easily accessible surface for the patient to pinch (e.g., with fingers, with a tool, etc.) relative to the insert. In another exemplary embodiment, the material of the nasal splint at extended portion 602 is thicker or bulkier than the rest of the nasal splint, advantageously providing yet further rigidity for supporting the splint during insert removal. Yet further, in embodiments of the nasal splint without extended portion 602, the material forming the anterior end of the splint (i.e., that will be the closest to the end of a user's nose when the splint is installed) may be made bulkier or thicker to provide improved installation and removal rigidity or gripping.

While support 604 is shown as embedded within the material of nasal splint 600, support 604 can be affixed, bonded, adhered, sutured, or otherwise coupled to an exterior surface of splint 600. Further, while one material for support 604 is aluminum, support 604 may be formed from alternative materials such as varying biocompatible silicones, polymers, other metals, plastics, or the like. Yet further, while support 604 is shown as being a long strip extending down the length of nasal splint 600, support 604 may include a plurality of smaller structures running at least partially across the length of nasal splint 600. In an exemplary embodiment the strip is thin such that it is not as thick as the walls of the nasal splint. For example, a series of small aluminum pieces may be arranged at an angle relative to the length of nasal splint 600 and provide supporting rigidity intended to resist buckling or other deformation of nasal splint 600 when the nasal splint insert is removed. Yet further, support 604 may be a thickened area of the septum plate of nasal splint 600 (e.g., double the thickness of the rest of splint 600) intended to provide increased lengthwise rigidity to splint 600.

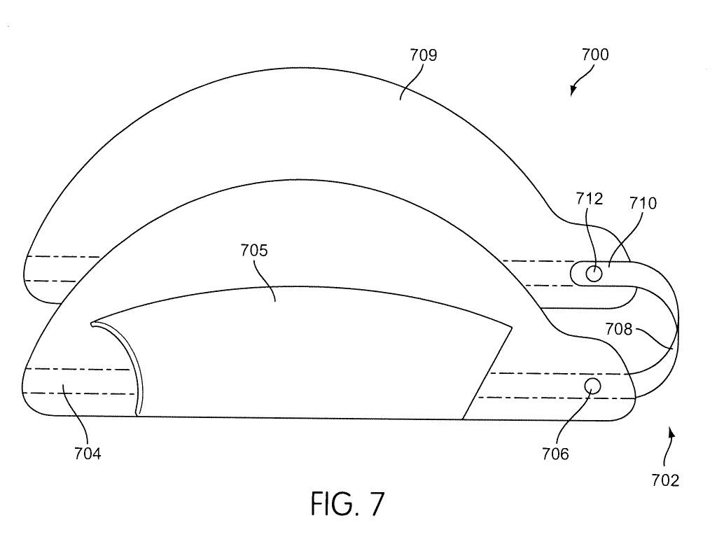

Referring now to FIG. 7, an improved nasal splint system 700 for use with a nasal splint insert such as insert 104 is shown, according to an exemplary embodiment. System 700 includes a strap 708 extending from an end portion 702 of a first nasal splint. The strap 708 can be used to provide a patient with an additional structure to grip when removing an insert from the first nasal splint's airway tube 705. For example, the patient can pinch, grab or push up on strap 708 as it extends from his or her nostril and as the patient is pulling out and down on a nasal splint insert to be removed from airway tube 705. In the embodiment shown in FIG. 7, the end portion 702 is at the anterior end of the nasal splints (i.e., the end that is closer to the front of the patient's nose).

Referring still to FIG. 7, nasal splint system 700 is also shown to include a second nasal splint 709 for insertion into a patient's left nasal cavity. Nasal splint 709 may be the same as splint 600 shown in FIG. 6 (e.g., having a support that prevents the tube from buckling and a hole 712 in an extended portion of the splint). Strap 708 may be integrally formed from end portion 702 of the first nasal splint but not integrally formed from the second nasal splint 709. In other embodiments, however, strap 708 may be integrally formed with both the nasal splint for the right nasal cavity and the nasal splint for the left nasal cavity. As shown in FIG. 7, strap 708 may include a hole 712 at or near the end 710 of the strap. Hole 712 may be used to suture or otherwise couple strap 708 to splint 709. For example, strap 708 may be sutured to splint 709 via hole 712.

When strap 708 is coupled to both nasal splints as shown in FIG. 7, strap 708 is shown to form a partial ring that may be convenient and easy for a user to push up on or grip while removing an insert. Strap 708 may be formed as a partial band by default. In other embodiments, the default shape for strap 708 may be relatively straight. In embodiments where strap 708 is relatively straight it may be formed from a material that is flexible enough to be bent for joining with a second nasal splint 709 as shown in FIG. 7. For example, strap 708 may be formed from the same semi-rigid material (e.g., a thin aluminum strip) as support 704 and bent into a partial ring shape (e.g., for joining with the second nasal splint 709).

Strap 708 may be integrally formed from support 704. In other embodiments, support 704 is formed from one material while the material of strap 708 is formed from a second material. For example, support 704 may be formed from aluminum and embedded within the nasal splint while strap 708 is formed from the same medical grade silicon as the rest of the splint and integrally formed from the extended portion of the splint. Yet further, while strap 708 is shown as being a relatively flat band of material, in other embodiments strap 708 may be round, oval, or any other shape. For example, in one exemplary embodiment, strap 708 may be a thick and round silicon tube. Such a structure and material for strap 708 may provide a relatively substantial structure for gripping during installation or removal of nasal splint inserts. For example, in one embodiment of the strap, the medical grade silicon may be thick enough to retain a ring-like shape and to be relatively rigid for a user to push up on when removing a nasal splint insert. A hole 706 may be provided in strap 708, for example, for suturing the splint to a patient's nose or to other stabilizers, grips, straps or other structures.

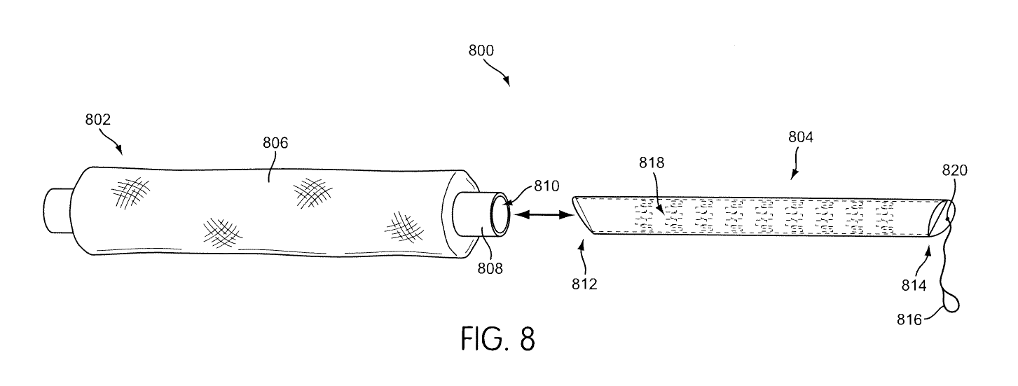

Referring now to FIG. 8, a nasal pack system 800 is shown, according to an exemplary embodiment. Nasal packs are used when an operation or event requires tamponading of nasal cavity vessels. For example, in an epistaxis/nose bleed scenario, nasal pack 802 may be installed to tamponade the nose bleed using packing material. Packing material 806 may be or include highly absorbent foam or any other soft, pliable, or absorbent material. Some nasal packs include an airway tube 808 as shown in FIG. 8. Airway tube 808 can become clogged with dried blood or mucus. Insert 804 is sized and shaped to be inserted into airway tube 808. More particularly, a first end 812 of insert 804 is inserted into opening 810 in airway tube 808. Insert 804 can be as described in varying embodiments described above. As shown, insert 804 is an elongated tube having a length and diameter for fitting within airway tube 808. Insert 804 provides an airway passage for a patient when the insert is installed within airway tube 808. When a blockage of blood or other human material forms within insert 804, insert 804 may be removed from airway tube 808. Insert 804 includes a plurality of protrusions 818 for carrying the obstruction with insert 804 when the insert is removed from airway tube 808. A second end 814 of insert 804 can include a tab or extended portion and a hole 820. Hole 820 may be used to suture or otherwise secure insert 804 to nasal pack 802, another insert, another nasal pack, the patient's nose, a strap as described above with reference to FIG. 7, or otherwise. In FIG. 8, the hole 820 is coupled to a pull 816 (e.g., a string with a loop) for facilitating removal of insert 804 from airway tube 808 by a patient or healthcare professional. Some nasal packs can be sized and shaped for sinus cavities and may include airway tubes. Inserts as described herein (e.g., having a plurality of protrusions for carrying human material) may be sized and shaped to fit such nasal packs.

The inserts and nasal splints described herein may be used with a method for treating a patient. The method includes inserting a nasal splint into a patient's nose, the nasal splint including an airway tube. The method further includes inserting an insert into the airway tube, the insert including an elongated tube and providing an airway passage for the patient within the airway tube. The method also includes allowing an obstruction of human material to form within the airway passage. The method yet further includes removing the insert from the nasal splint and the patient's nose to remove the obstruction of human material from the patient's nose with the insert. The elongated tube may include a plurality of protrusions for carrying the obstruction with the insert when the insert is removed from the airway tube of the nasal splint. The method may further include inserting another insert into the nasal splint after the insert with the obstruction of human material is removed from the nasal splint. It may be possible to repeat the insertion and removing steps while human material continues to be collected by the inserts. For example, an insert may be removed from the nasal splint each time it becomes apparent that the insert is full of human material. The inserts may be removed and replaced (with a new insert) according to a schedule (e.g., each night prior to planned sleeping periods, each morning, one day after surgery, etc.). Once bleeding or drainage of nasal fluids subsides, a final insert may be installed into the nasal splint (e.g., to catch any remaining bleeding or drainage) or the nasal splint may be left empty without an insert. The reinsertion step may be facilitated by a lubricant or jelly. In other embodiments, the exterior surface of the insert may have a coefficient of friction such that a lubricant or jelly is not necessary.

The construction and arrangement of the exemplary systems and methods as shown or described are illustrative only. Although only a few embodiments have been described in detail in this disclosure, many modifications are possible (e.g., variations in sizes, dimensions, structures, shapes and proportions of the various elements, values of parameters, mounting arrangements, use of materials, orientations, etc.). For example, the position of elements may be reversed or otherwise varied and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions and arrangement of the exemplary embodiments without departing from the scope of the present disclosure.

What is claimed is:

1. A system comprising:
a nasal splint comprising an airway tube; and
an insert comprising an elongated tube for insertion into the airway tube of the nasal splint;
wherein the elongated tube of the insert is configured to collect an obstruction of human material while installed in the airway tube and to remove the obstruction when the insert is removed from the airway tube.

2. The system of claim 1, wherein the nasal splint further comprises a strap extending from one end of the nasal splint and for gripping by a patient when the insert is being removed from the airway tube.

3. The system of claim 2, wherein the nasal splint further comprises a support extending along the length of the airway tube, the support adding rigidity to the nasal splint.

4. The system of claim 3, further comprising:
a second nasal splint for insertion into a second nasal cavity;
wherein the strap is configured for coupling to the second nasal splint.

5. A device for use with a nasal splint having an airway tube, comprising:
an elongated tube for insertion into the airway tube of the nasal splint, the elongated tube having an exterior surface that presses against the airway tube and an interior surface that forms an airway passage; and
a plurality of protrusions extending from the interior surface of the elongated tube and into the airway passage.

6. The device of claim 5, wherein the elongated tube is at least as long as the airway tube of the nasal splint.

7. The device of claim 5, wherein the elongated tube is a semi-cylindrical shape having a flat portion and a curved portion.

8. The device of claim 7, wherein the plurality of protrusions extend from the interior surface corresponding with at least one of the flat portion of the elongated tube and the curved portion of the elongated tube.

9. The device of claim 7, further comprising:
a tab extending from the flat portion of the elongated tube and for gripping by a patient.

10. The device of claim 5, wherein the plurality of protrusions are arranged in one or more columns down the length of the elongated tube, one or more rows across the length of the elongated tube, or are arranged in at least one column down the length of the elongated tube and are arranged in at least one row across the length of the elongated tube.

11. The device of claim 5, wherein the plurality of protrusions are staggered.

12. The device of claim 5, wherein the plurality of protrusions comprise at least one of: hooks, loops, bristles, spikes, a raised matrix, a raised grid, and combinations thereof

13. A system comprising:
a nasal splint; and
an insert for insertion into a patient's nose with the nasal splint, the insert having an elongated tube for providing the patient with an airway passage;
wherein the elongated tube is configured to remove an obstruction of human material from the patient's nose when the insert is removed from the patient's nose.

14. The system of claim 13, wherein the nasal splint further comprises a support extending along the length of the nasal splint and adding rigidity to the nasal splint.

15. The system of claim 14, wherein the nasal splint comprises a flexible body and wherein the support is at least partially embedded within the body.

16. The system of claim 15, wherein the support is fully embedded within the body.

17. The system of claim 15, wherein the support is coupled to the exterior of the flexible body.

18. The system of claim 15, wherein the support comprises a strip of aluminum.

19. The system of claim 15, wherein the support comprises a rigid piece of polymer-based material.

20. The system of claim 15, wherein the nasal splint comprises an extended portion that projects from one end of the nasal splint.

21. The system of claim 20, wherein the support is coupled to the extended portion.

22. The system of claim 21, wherein the nasal splint further comprises a strap projecting from the extended portion and for gripping by a patient when the insert is being removed.

23. The system of claim 22, wherein the support prevents the body of the nasal splint from buckling when the strap is gripped and the insert is being pulled out of the nasal splint.

24. The system of claim 23, further comprising:
a second nasal splint for insertion into a second nasal cavity;
wherein the strap is configured for coupling to the second nasal splint.

25. The system of claim 13, further comprising:
at least one replacement insert for inserting into the airway passage after a first insert has been removed from the airway passage of the nasal splint.

26. A method for treating a patient having a nasal splint installed in his or her nose, the nasal splint including an airway tube, the method comprising:
inserting an insert into the airway tube, the insert comprising an elongated tube and providing an airway passage for the patient within the airway tube, wherein the insert is configured to remove an obstruction of human material from the airway tube when the insert is removed from the airway tube.

27. The method of claim 26, wherein the elongated tube includes a plurality of protrusions for carrying the obstruction with the insert when the insert is removed from the airway tube of the nasal splint.

28. The method of claim 27, further comprising:
inserting another insert into the nasal splint after the insert with the obstruction of human material is removed from the nasal splint.

29. The method of claim 26, wherein the method further comprises inserting the nasal splint into a patient's nose.

30. The method of claim 26, further comprising:
allowing the obstruction of human material to form within the airway passage; and
removing the insert from the nasal splint and the patient's nose to remove the obstruction of human material from the patient's nose with the insert.

31. An insert for use with a nasal splint, the insert comprising:
an elongated tube for insertion into a nasal splint; and
a plurality of protrusions extending from the interior surface of the elongated tube and into an airway passage formed by the elongated tube.

32. A system comprising:
a nasal pack having an airway tube; and
an insert comprising an elongated tube for insertion into the airway tube of the nasal pack, the elongated tube having an exterior surface that presses against the airway tube and an interior surface that forms an airway passage, wherein the insert further comprises a plurality of protrusions extending from the interior surface of the elongated tube and into the airway passage.
